SEQUENCE LISTING (6 sequences)

SEQ ID NO: 1 - DNA, 27 bp, artificial sequence
  synthetic oligonucleotide primer
gcatcatacaagcttggtaccnnntct

SEQ ID NO: 2 - DNA, 28 bp, artificial sequence
  synthetic oligonucleotide primer
gcatcatacaagcttggtaccnnnntct

SEQ ID NO: 3 - DNA, 29 bp, artificial sequence
  synthetic oligonucleotide primer
gcatcatacaagcttggtaccnnnnntct

SEQ ID NO: 4 - DNA, 30 bp, artificial sequence
  synthetic oligonucleotide primer
gcatcatacaagcttggtaccnnnnnntct

SEQ ID NO: 5 - DNA, 40 bp, artificial sequence
  synthetic oligonucleotide primer
actaatacgactcactataggttttttttttttttttttv

SEQ ID NO: 6 - DNA, 21 bp, artificial sequence
  synthetic oligonucleotide primer
gcatcatacaagcttggtacc

(19) United States
(12) Patent Application Publication
(10) Pub. No.: US 2006/0035226 A1
(43) Pub. Date: Feb. 16, 2006

(54) Amplification of ribonucleic acids

(76) Inventors: Peter Scheinert, Hamburg (DE); Guido Krupp, Gnutz (DE)

Correspondence Address: ARNOLD & PORTER LLP;ATTN: IP DOCKETING DEPT., 555 TWELFTH STREET, N.W., WASHINGTON, DC, 20004-1206, US

(21) Appl. No.: 10/516,007

(22) PCT Filed: May 27, 2003
(86) PCT No.: PCT/EP03/05579

§ 371 Date: Jun. 8, 2005

(30) Foreign Application Priority Data
May 31, 2002 (DE) 102 24 200.3

(51) Int. Cl. C12Q 1/68; C12P 19/34

(52) U.S. Cl. 435/6; 435/91.2

(57) ABSTRACT

The invention relates to methods for the amplification of ribonucleic acids, comprising the following steps: (a) a single stranded DNA is produced from an RNA by means of reverse transcription, using a single-stranded primer having a defined sequence, an RNA-dependent DNA polymerase and deoxyribonucleoside triphosphates; (b) the template RNA is removed; (c) a DNA duplex is produced by means of a single-stranded primer comprising a box sequence, a DNA polymerase and deoxyribonucleoside triphosphates; (d) the duplex is separated into single-stranded DNAs; (e) DNA duplexes are produced from one of the single-stranded DNAs obtained in step (d) by means of a single-stranded primer comprising a promoter sequence at its 5′end and the same defined sequence as the primer used in step (a) at its 3′end, a DNA polymerase and deoxyribonucleoside triphosphates; (f) a plurality of RNA single strands, both ends of which comprise defined sequences, are produced by means of an RNA polymerase and ribonucleoside triphosphates. The invention also relates to kits for amplifying ribonucleic acids according to one of said methods, said kits comprising the following components: (a) at least at least one single-stranded primer, which contains a promoter sequence; (b) at least one single-stranded primer comprising a box sequence; (c) an RNA-dependent DNA polymerase; (d) deoxyribonucleoside triphosphates; (e) a DNA-dependent DNA polymerase; (f) an RNA polymerase; and (g) ribonucleoside triphosphates.

### CROSS-REFERENCE TO RELATED APPLICATIONS

This application is a national phase application under 35 U.S.C. § 371 of International Application Number PCT/EP03/05579, filed May 27, 2003, the disclosure of which is hereby incorporated by reference in its entirety, and claims the benefit of German Patent Application Number 102 24 200.3, filed May 31, 2002.

### INCORPORATION OF SEQUENCE LISTING

A paper copy of the Sequence Listing and a computer readable form of the sequence listing on diskette, containing the filed named “SL19006004.txt”, which is 1,943 bytes in size (measured in MS-DOS), and which was recorded on Nov. 29, 2004, are herein incorporated by reference.

### BACKGROUND OF THE INVENTION

To date, a multitude of processes resulting in the amplification of nucleic acids are known. The best known example is the polymerase chain reaction (PCR), developed by Kary Mullis in the mid-eighties (see Saiki et al., Science, Vol. 230 (1985), 1350-1354; and EP 201 184).

During the PCR reaction, single-stranded primers (oligonucleotides with a chain-length of usually 12 to 24 nucleotides) bind to a complementary, single-stranded DNA sequence. These primers are subsequently elongated to double stranded DNA, in the presence of a DNA polymerase and deoxyribonucleoside triphosphates (dNTPs, namely dATP, dCTP, dGTP and dTTP). The double stranded DNA is separated by heating into single strands. The temperature is reduced sufficiently to allow a new step of primer binding. Again, primer elongation results in double stranded DNA.

Repetition of the steps described above enables exponential amplification of the input DNA. This is achieved by adjusting the reaction conditions such that almost each molecule of single-stranded DNA within each round of amplification will be transformed into double stranded DNA, melted into single-stranded DNAs which will be used again as template for the next round of amplification.

It is possible to conduct a reverse transcription reaction prior to the above mentioned PCR reaction. This means, in the presence of an RNA-dependent DNA polymerase mRNA is transformed into single-stranded DNA (cDNA), which can then be used in a PCR reaction, hence resulting in the amplification of RNA sequences (see EP 201 184).

This basic reaction model of a PCR reaction has been altered in the last years and a multitude of alternatives have been developed, depending on the starting materials (RNA, DNA, single or double stranded) and also relating to different reaction products (amplification of specific RNA or DNA sequences from the mixture of different nucleic acids within one sample, or the amplification of all RNA/DNA sequences present in one sample).

Over the last years, so called microarrays for the analysis of nucleic acids are used with increasing frequence. The essential component of such a microarray is an inert carrier onto which a multitude of different nucleic acid sequences (mostly DNA) were bound in different regions of the carrier. Usually, within one particular very small region, only DNA with one specific sequence is bound, resulting in microarrays with several thousand different regions capable of binding several thousand different sequences.

These microarray plates can be incubated with a multitude of nucleic acid sequences (mostly also DNA) obtained from a sample of interest. Resulting, under suitable conditions (ion content, temperature and so forth), in complementary hybrid molecules of nucleic acid sequences from those sequences originating form the sample of interest and those sequences bound to the microarray plate. Unbound, non-complementary sequences can be washed off. The regions on the microarray containing double stranded DNA can be detected and thus, the sequences as well as the amount of nucleic acids bound from the original sample can be analysed.

Microarrays are used to analyse expression profiles of cells, hence allowing the analysis of all mRNA sequences expressed in certain cells (see Lockhart et al., Nat. Biotechnol. 14 (1996), 1675-1680).

The amount of mRNA available for this sort of analysis is usually limited. Therefore special processes have been developed to amplify the ribonucleic acids, which will be analysed by means of microarrays. To this end, ribonucleic acids will possibly be converted to more stable cDNAs by means of reverse transcription.

Methods, yielding large amounts of amplified RNA populations of single cells are described in e.g. U.S. Pat. No. 5,514,545. This method uses a primer containing an oligo-dT-sequence and a T7-promoter region. The oligo-dT-sequence binds to the 3′-poly-A-sequence of the mRNA initiating the reverse transcription of the mRNA. Alkaline conditions result in the denaturation of the RNA/DNA heteroduplex, and the hairpin structure at the 3′-end of the cDNA can be used as primer to initiate synthesis of the second DNA strand. The resulting construct is converted to a linear double stranded DNA by using nuclease S1 to open the hairpin structure. Then the linear double stranded DNA is used as template for T7 RNA polymerase. The resulting RNA can be used again as template for the synthesis of cDNA. For this reaction oligonucleotide hexamers of random sequences (random primers) are used. Following heat-induced denaturation, the second DNA strand is produced by means of the above mentioned T7-olido-dT-primer and the resulting DNA can again be used again as template for T7 RNA polymerase.

An alternative strategy is presented in U.S. Pat. No. 5,545,522. Here, it is demonstrated that a single oligonucleotide primer can be used to yield high amplifications. RNA is reverse transcribed to cDNA, and the primer has the following characteristics: a) 5′-dN₂₀, meaning a random sequence of 20 nucleotides; b) a minimal T7-promoter; c) GGGCG as transcription-initiation sequence; and d) oligo-dT₁₅. Synthesis of the second DNA strand is achieved by partial RNA digestion by RNase H. The remaining RNA-oligonucleotides are used as primers for DNA polymerase I. The ends of the resulting DNA are blunted by T4-DNA polymerase.

A similar procedure is disclosed in U.S. Pat. No. 5,932,451. In this procedure, two so-called box-primers are added within the 5′ proximal area, enabling the double immobilisation by using biotin-box-primers.

However, the above mentioned methods to amplify ribonucleic acids have major disadvantages. All of the above mentioned methods result in RNA populations which are different from the RNA populations present in the original starting material. This is due to the use of the T7-promoter-oligo-dT-primers, which do primarily amplify RNA sequences of the 3′-section of the mRNA. Furthermore, it has been shown that those extremely long primers (more than 60 nucleotides) are prone to build primer-primer-hybrids and they do also allow for non-specific amplification of the primers (Baugh et al., Nucleic Acids Res. 29 (2001) E29). Therefore the known procedures result in the production of a multitude of artefacts, interfering with the further analysis of the nucleic acids.

The problem underlying the present invention therefore resides in providing a method to amplify ribonucleic acids, which allows homogeneous and in particular highly reproducible amplification of the ribonucleic acids present in the starting material.

This problem is now solved using a method comprising the following steps:
a) a single stranded DNA is produced from an RNA by means of reverse transcription, using a single-stranded primer having a defined sequence, an RNA-dependent DNA polymerase and deoxyribonucleoside triphosphates;
b) the template RNA is removed;
c) a DNA duplex is produced by means of a single-stranded primer comprising a box sequence, a DNA polymerase and deoxyribonucleoside triphosphates;
d) the duplex is separated into single-stranded DNAs;
e) DNA duplexes are produced from one of the single-stranded DNAs obtained in step (d) by means of a single-stranded primer comprising a promoter sequence at its 5′end and the same defined sequence as the primer used in step (a) at its 3′end, a DNA polymerase and deoxyribonucleoside triphosphates; and
f) a plurality of single stranded RNAs is produced, both ends of which comprise defined sequences, by means of an RNA polymerase and ribonucleoside triphosphates.

### BRIEF SUMMARY OF THE INVENTION

The present invention includes a process for the amplification of ribonucleic acids, comprising the following steps: (a) a single stranded DNA is produced from an RNA by means of reverse transcription, using a single-stranded primer having a defined sequence, an RNA-dependent DNA polymerase and deoxyribonucleoside triphosphates; (b) the template RNA is removed; (c) a DNA duplex is produced by means of a single-stranded primer comprising a box sequence, a DNA polymerase and deoxyribonucleoside triphosphates; (d) the duplex is separated into single-stranded DNAs; (e) DNA duplexes are produced from one of the single-stranded DNAs obtained in step (d) by means of a single-stranded primer comprising a promoter sequence at its 5′end and the same defined sequence as the primer used in step (a) at its 3′end, a DNA polymerase and deoxyribonucleoside triphosphates; and (f) a plurality of single stranded RNAs is produced, both ends of which comprise defined sequences, by means of an RNA polymerase and ribonucleoside triphosphates. The present invention further provides kits for amplifying ribonucleic acids according to one of said processes, said kits comprising the components required for performing the processes of the present invention.

### BRIEF DESCRIPTION OF THE DRAWINGS

FIG. 1 sets forth a schematic diagram of the process of the present invention.

### DESCRIPTION OF THE NUCLEIC ACID SEQUENCES

SEQ ID NO: 1 sets forth a nucleic acid sequence of a primer of the present invention.
SEQ ID NO: 2 sets forth a nucleic acid sequence of a primer of the present invention.
SEQ ID NO: 3 sets forth a nucleic acid sequence of a primer of the present invention.
SEQ ID NO: 4 sets forth a nucleic acid sequence of a primer of the present invention.
SEQ ID NO: 5 sets forth a nucleic acid sequence of a primer of the present invention.
SEQ ID NO: 6 sets forth a nucleic acid sequence of a primer of the present invention.

### DETAILED DESCRIPTION OF THE INVENTION

It was surprisingly found that the above combination of steps leads to a remarkably homogeneous amplification of the ribonucleic acids present in the starting material. At the same time the process according to the invention prevents the production of artefacts. Hence the process according to the invention is a substantial improvement of methods to amplify nucleic acids and allows at the same time the improvement of procedures to analyse ribonucleic acids by means of microarrays.

The process according to the invention results in the amplification of single-stranded ribonucleic acids which have the inverse orientation (antisense sequence) when compared to the ribonucleic acids present in the starting material.

According to one embodiment of the processes of the invention, the ribonucleic acids used as template in step (a) were isolated from cells of an organism. The isolation of mRNA from cells of multi-cellular organisms is especially preferred.

The single-stranded primer used in (a) is a primer of any defined sequence, this mans that this primer does not contain a random sequence of nucleotides. Also more than one defined primer can be used.

The single-stranded primer described in (a) contains preferably an oligo-dT-sequence, a sequence containing several dT-nucleotides. This has the advantage that the primer binds to the poly A-tail of eukaryotic mRNA. This results in reverse transcription of almost exclusively mRNA.

In the process according to the invention it is preferred if the primer described in (a) contains a 5′-(dT)₁₈V sequence. This is a primer with 18 dT-deoxyribonucleotide-monomers followed by a single deoxyribonucleotide of different nature (namely dA, dC, or dG, here referred to as V). This primer nearly exclusively allows reverse transcription of sequences which are located in the close vicinity of the 5′-end of the polyA-tail. Different to known processes, the use of such a primer therefore suppresses the production of artefacts resulting from further downstream primer binding of normal oligo-dT-primers within the large polyA-areas of mRNAs.

Alternatively, the primer of (a) can be homologous to one or several specific sequences, present in the sample. In this procedure, the amplification of ribonucleic acids is limited to specific target sequences.

Further, in the process according to the invention, it is preferred if the RNA-part of the DNA-RNA-hybrids described in (b) is digested by RNase. For this procedure any RNase can be used. The use of RNase I and/or RNase H is preferred. This step results in the elimination of all RNAs which have not been transcribed into cDNA during the first step of the procedure, particularly ribosomal RNAs, but also all other cellular RNAs which do not have the polyA-tail, characteristic for mRNAs.

The DNA-RNA-hybrids, which result from the reverse transcription reaction can also be separated into single strands by means of heat. However, different to heat treatment, the use of RNases has the further advantage that genomic DNA present in the sample is not converted to single-stranded form, and thus it will not act as a hybridisation template for the primers used in the following steps of the procedure. Special advantages result from the use of RNase I, because this enzyme can easily be inactivated at temperatures below those resulting in denaturation of the genomic DNA. The aim of the process according to the invention is the amplification of ribonucleic acids, hence the use of a stable RNase could hinder this process and would necessitate elimination by elaborate procedures.

In step (c) a single-stranded primer is used, which contains a Box sequence. Within the scope of the present invention an RNA- or DNA-sequence is called a Box sequence if it comprises a defined sequence of 10 to 25 nucleotides, having only low homology to gene sequences of the organisms from which the starting RNA template for amplification was isolated from.

Low homology between a potential Box sequence and corresponding gene sequences can be determined experimentally by means of standard Northern Blot analysis. To this end RNA samples from an organism of interest (e.g. plants, humans or animals), this means the organism from which RNA was isolated for further amplification, is separated by means of electrophoresis and transferred onto a membrane and hybridised with a labelled oligonucleotide containing the Box sequence. Low homology is characterised by the absence of a hybridisation signal under stringent hybridisation conditions. For example, stringent conditions can be achieved by washing the membrane, after the hybridisation, for 40 minutes at 25° C. with a buffer containing 0.1*SSC and 0,1% SDS.

As an alternative to the above mentioned experimental procedure to verify a Box sequence, it is possible to determine a sequence with low homology by searching databases containing known gene sequences, that are expressed in multi-cellular organisms. To date, all known gene sequences that are expressed in multi-cellular organisms are stored in databases with open access to the public. These sequences are either stored as gene sequences with known function, or if the function is not known as so called “expressed sequence tags” or ESTs. A sequence with only low homology to known sequences is suitable as a Box sequence, if this sequence in comparison to all sequences listed in a database, shows over a total length of 10 to 25 nucleotides at least 20%, but preferably 30 or 40%, differences in their sequences. This means that over a length of 10 nucleotides at least 2 nucleotides are different, and 4 over a length of 20 nucleotides, respectively. Sequence identities, or differences between 2 sequences are preferably determined using the BLAST software.

Therefore, a certain sequence can be determined as a Box sequence for a certain use. If human mRNA is to be amplified in the process according to the invention, the described low homology has to be determined by comparison with a human database or hybridising human RNA with the Box sequence in a Northern Blot. If plant mRNA is to be amplified in the process according to the invention, the described low homology has to be determined by comparison with plant ribonucleic acids. A sequence, suitable as a Box sequence, in a certain uses of the process according to the invention, therefore might not be suitable as Box sequence in a different use.

The Box sequence is preferably selected not to contain viral sequences, neither coding nor regulatory sequences (promoter or terminator sequences) of viruses or bacteriophages.

In the process according to the invention, use of a primer comprising a suitable Box sequence is highly advantageous, because this drastically reduces the production of amplification artefacts.

The Box sequence is located in the 5′ region of the primer used in step (c). Preferably the primer further contains a sequence of 3 to 6 random nucleotides (N-3-N6), and a defined trinucleotide sequence (for example TCT). Alternatively, a mix of different trinucleotide sequences can be included in the primer.

Preferably, the primer containing the Box sequence has a length of 40 nucleotides, a length of 30 nucleotides is especially preferred.

In an especially preferred version of the process according to the invention, a single-stranded primer is used in step (c) comprising in addition to the Box sequence an especially suitable sequence of at least 6 nucleotides. The primer to be used in step (c) can, for example, have the following sequence: GCA TCA TAC AAG CTT GGT ACC N₃₋₆ TCT (27-30 nt).

In steps (c) and (e), any DNA-dependent DNA polymerase can be used. Preferably, a reverse transcriptase is used. It is especially advantageous in the process according to the invention, to use a reverse transcriptase, because this DNA polymerase does not separate double stranded DNA. For the DNA polymerisation in steps (a), (c) and (e) also deoxyribonucleoside triphosphates are needed, usually dATP, dCTP, dGTP and dTTP.

In step (d), separation of double stranded DNA into single strands can be achieved by any procedure. However, this is preferably done by means of heat.

In step (e) a single-stranded primer is used, which contains a promoter sequence. A promoter sequence allows the binding of the RNA polymerase and initiates the synthesis of an RNA strand. Preferred in (e) is the use of a single-stranded primer containing the sequence of a highly specific RNA polymerase promoter like T7, T3 or SP6. A primer with a T7-promoter has for example the following sequence: ACT AAT ACg ACT CAC TAT A g⁺¹ g (dT)₁₈V (40 nt).

Selecting an RNA polymerase to be used in the method of the present invention in step (f) depends on the promoter sequence used in the primer sequence. If the primer contains a T7 polymerase sequence, then a T7 RNA polymerase has to be used in step (f).

To obtain ribonucleic acids in step (f), ribonucleotide-monomers are further needed, usually ATP, CTP GTP and UTP.

For the first time, the process according to the invention allows a strong and specific amplification of the starting RNA sequences, representing the total sequences of the entire RNA population. The amplification factor of the starting RNA sequence is at least 500, whereas a factor of more than 1000 is especially preferred.

The present invention also includes processes according to the invention, which result in removal of single-stranded primers and primer induced artefacts (e.g. primer-dimers), before the RNA polymerase is added.

Further amplification of ribonucleic acids can be achieved in processes wherein the following steps are performed after step (f):
(g) single-stranded RNAs generated in step (f) are used as a template to synthesize single-stranded DNA by means of reverse transcriptase, a single-stranded primer, containing the Box sequence, an RNA-dependant DNA polymerase and deoxyribonucleoside triphosphates;
(h) the RNA is removed;
(i) using the in (h) generated single-stranded DNA as template, double-stranded DNA is synthesised using a single-stranded primer, comprising a promoter sequence in its 5′ region and the same defined sequence as the primer used in step (a), in its 3′ region, a DNA polymerase and deoxyribonucleoside triphosphates;
(j) a multitude of single-stranded RNAs are synthesized using a RNA polymerase and ribonucleoside triphosphates.

This variation of the process according to the invention has specific advantages. The defined sequence at the ends of the ribonucleic acids, produced in steps (a) to (f) in the process facilitates reverse transcription into DNA. This DNA can be used as template for further, promoter-based RNA synthesis. In this manner, a further at least 50-fold increase of the amount of amplified ribonucleic acids can be achieved.

Preferably the process according to the invention is performed such that the single-stranded primer used in step (i) has the same sequence as the primer used in step (a).

The primer used in step (g) can be identical to the primer used in step (c). Alternatively, the primer used in step (g) can consist only of the well defined Box sequence, and does not include the less specific elements of the primer used in step (c). The primer used in step (g) can, for example, have the following sequence: GCA TCA TAC AAG CTT GGT ACC (21 nt).

The RNA produced in step (h) can be removed with any known, appropriate procedure, however, hydrolysis with RNase is preferred.

Before proceeding with the transcription reactions (steps (f) and (j) according to the process of the invention) it may be advantageous to use any known procedures for purifying the nucleic acids thus generated. During such a purification procedure, special care should be taken that any excess of primers and/or primer induced artefacts (e.g. primer dimers) are removed.

The process according to the invention as described above produces exclusively single-stranded RNA with antisense orientation (so called antisense strands). The present invention also covers process, which by means of to date known standard processes (reverse transcription, PCR, cDNA second-strand synthesis, transcription and so forth) convert the single-stranded antisense RNA into double-stranded DNA, single-stranded DNA of any orientation, or into single-stranded RNA with sense-orientation (so called sense-strand). The precise manner of the procedure and the resulting product is highly dependant on the intended use.

The present invention also relates to kits comprising all reagents needed to amplify ribonucleic acids by means of the process according to the invention. These kits kits may comprise the following components:
(a) at least one single-stranded primer, comprising a promoter sequence;
(b) at least one single-stranded primer, comprising a Box sequence
(c) an RNA-dependent DNA polymerase;
(d) deoxyribonucleoside triphosphates;
(e) a DNA-dependent DNA polymerase;
(f) an RNA polymerase; and
(g) ribonucleoside triphosphates

Accordingly, the kit contains at least two different single-stranded primers, which are characterised by the above mentioned criteria. However, dependent on the intended use, the kit may contain more than two primers and additional reagents.

In addition, the kit may contain RNase I and/or RNase H.

The kit contains a DNA polymerase, preferably a reverse transcriptase or any other DNA polymerase, which does not separate double-stranded DNA.

The kit may further comprise a composition for DNA-labelling with a detectable moiety and one or more DNA microarrys. The kit may thus contain all components necessary to perform gene expression analysis. In general, the different components of the kit will be supplied in different tubes. However, components used in the same step of the procedure may also be supplied in one tube.

Therefore, the present invention further relates to procedures for the analysis of nucleic acids, during which ribonucleic acids are obtained and amplified using any of the procedures described in the present invention and which will thereafter be analysed using a microarray technique. Ribonucleic acids are normally isolated form biological samples. Prior to microarray analysis, ribonucleic acids amplified by techniques described in the present invention might be transcribed into cDNA, using a reverse transcription. The present invention allows analysis of amount and/or sequence of the cDNA. The DNAs obtained in the intermediate steps can also be used, for example, to generate, by means of cloning, a representative genebank, containing genes derived from a biological sample or genes derived from a sample produced in a laboratory.

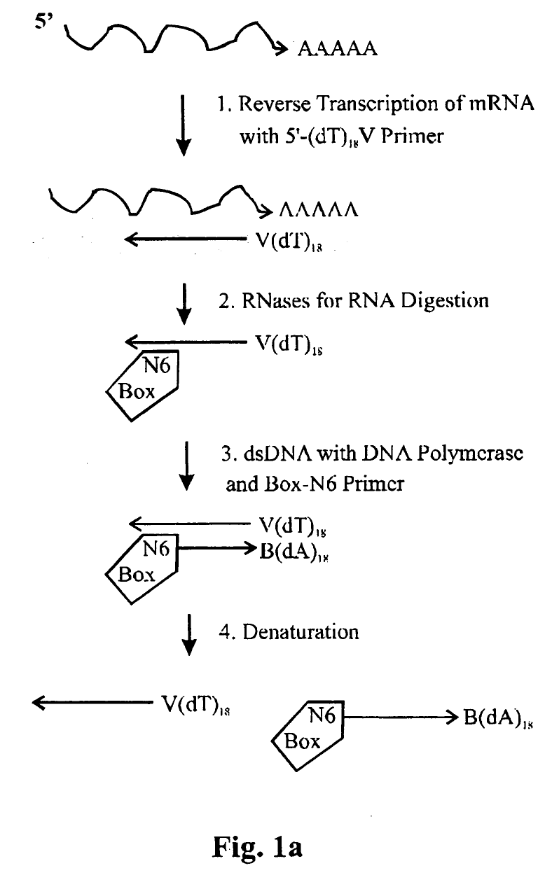

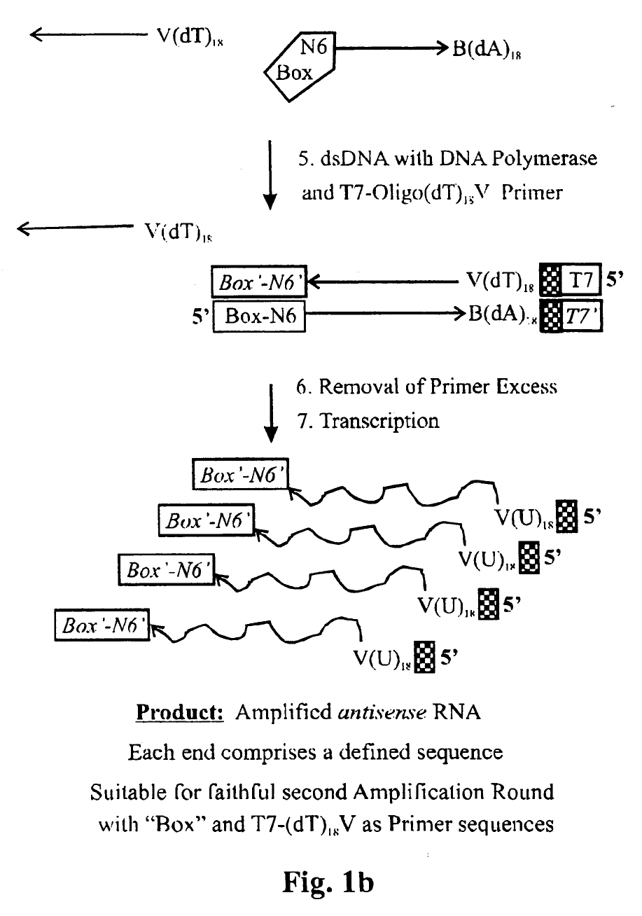

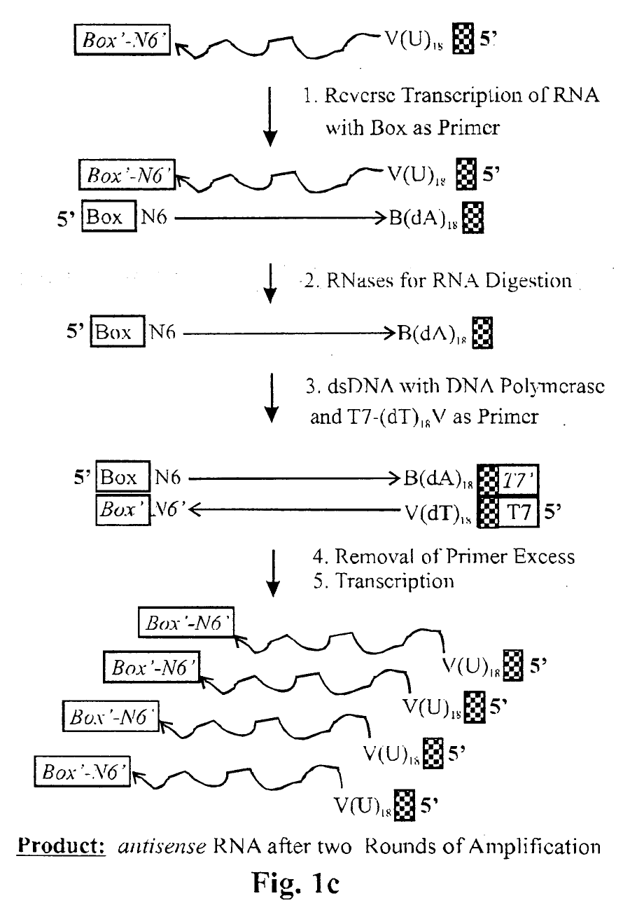

FIG. 1 illustrates an example of the procedures of the present invention as a schematic diagram: In a first step RNA is transcribed into single-stranded DNA by means of reverse transcription, using an anchored oligo(dT)₁₈V primer. This procedure allows the reverse transcription starting at the transition of the ploy-A tail of the mRNA to the 3′-UTR area. The next step eliminates the RNA from the RNA-cDNA-heteroduplex by use of RNase H/RNase I and the remaining RNA (mainly ribosomal RNA) is digested by RNase I.

Synthesis of the second, complementary DNA strand is used to introduce the Box sequence via a specific primer. This primer consists in one part of 6-9 random nucleotides and a second part which comprises the Box sequence.

After primer annealing, elongation to double stranded DNA is achieved by incubation with DNA polymerase. Excess primers are removed and heat-induced denaturation of the DNA double strand is followed by a reduction of the incubation temperature, enabling a primer containing the T7-promoter and a (dT)₁₈V sequence to hybridise with the DNA. A further DNA strand is obtained by primer elongation. Hereafter excess primer and primer-induced artefacts (primer dimers) are removed and the RNA amplification is achieved by in vitro transcription utilizing the T7 promoter.

FIG. 1c describes the procedure to amplify ribonucleic acids according to the above mentioned steps (g-j). The ribonucleic acid produced in step (f) is reverse transcribed, using a primer containing the Box sequence, a reverse transcriptase and dNTPs. RNases remove the RNA-strand. Using a primer, containing a promoter and the oligo-dT sequence, a second DNA strand is produced, that is used as template for the RNA polymerase in the transcription reaction.

The order and detailed implementation of the reaction steps of the present invention are illustrated by the Examples:

### EXAMPLES

### Example 1

### First amplification round (see, e.g., FIGS. 1a, 1b)

### Example 1A

### Reverse Transcription of 100 ng Total-RNA Using Oligo(dT)₁₈V-primer

| First strand-DNA-Synthesis: | |
|---|---|
| RNA (50 ng/μl): | 2 μl |
| Oligo(dT)₁₈ V(5 pmol/μl): | 1.5 μl |
| dNTP-Mix (10 mM): | 1 μl |
| DEPC-H₂O | 3.5 μl |

Incubate 4 min at 65° C. in a thermocycler with a heated lid, then place immediately on ice.

| Mastermix for synthesis of the 1^{st} strand of cDNA | |
|---|---|
| 5 × RT-buffer | 4 μl |
| RNase-inhibitor (20 U/μl) | 1 μl |
| Superscript II (200 U/μl) | 1 μl |
| DEPC-H₂O | 6 μl |

Pipette components for the mastermix on ice and add to the tube containing the reverse transcription mix. Place samples in a thermocycler (preheated to 42° C.)

Incubate as follows:
37° C./5 minutes
42° C./50 minutes
45° C./10 minutes
50° C./10 minutes
70° C./15 minutes (enzyme inactivation)

Place samples on ice.

### Example 1B

### RNA Removal

| Removal of RNA from the reaction | |
|---|---|
| First strand-cDNA mix | 20 μl |
| RNase-Mix (RNase H/RNase I; each at 5 U/μl) | 1 μl |

Incubate for 20 min at 37° C., hereafter place samples on ice. RNase A was not used for RNA elimination, because RNase A is not readily inactivated. RNase I on the other hand, the enzyme used in this invention, can be inactivated easily and completely by incubation at 70° C. for 15 min.

### Example 1C

### Double-Stranded Template DNA with Box-Random-Primer and T7-(dT)₁₈V

| Random priming of first strand cDNA with Box-random Primer | |
|---|---|
| First strand-cDNA | 21 μl |
| dNTP-mix (10 mM) | 1 μl |
| Box-random-primer (10 pmol/μl) | 1 μl |
| 10 × polymerase buffer | 6 μl |
| H₂O | 20 μl |

Incubation:
70° C./1 minute
37° C./1 minute
add 1 μl reverse transcriptase (5 U/μl) to each sample
incubate at 37° C./30 minutes

Removal of excess primer
1 μl Exonuclease I (10 U/μl)
37° C./5 minutes
96° C./6 minutes

Place samples on ice

Double-stranded template DNA with T7-(dT)₁₈V
2 μl T7-(dT)₁₈V primer (10 pmol/μl)
70° C./1 minute
42° C./1 minute
add 1 μl reverse transcriptase (5 U/μl) to each sample
42° C./30 minutes
cool to 37° C.
1 μl T4 DNA polymerase (10 U/μl)
37° C./1 minute
65° C./1 minute

Place samples on ice.

### Example 1D

### Purification of the cDNA with High-Pure PCR Purification Kit (Roche)

| cDNA purification | |
|---|---|
| Reaction mix | 50 μl |
| Binding-buffer | 250 μl |
| Carrier (cot-1-DNA, 100 ng/μl) | 3 μl |

Transfer mix onto provided columns, spin in a tabletop centrifuge at maximal rpm for 1 min. Discard the flow-through. Add 500 μl washing buffer to the column and spin as above, discard flow-through and repeat the wash step with 200 μl washing buffer. Transfer columns onto a new 1.5 ml reaction tube add 50 μl elution buffer, incubate for 1 min at RT and centrifuge as described above. Repeat the elution step once, again using 50 μl buffer.

### Example 1E

### Ethanol Precipitation of Purified cDNA

Do not vortex the Pellet Paint™-carrier stock solution and store in the dark. Keep at −20° C. for long term storage, smaller aliquots can be stored for approximately 1 month at 4° C.

| Ethanol precipitation | |
|---|---|
| Eluate | 100 μl |
| Carrier (Pellet Paint ™) | 2 μl |
| Sodium acetate | 10 μl |
| Ethanol; absolute | 220 μl |

Mix thoroughly (do not vortex) and pellet cDNA by centrifugation at maximal rpm for 10 min at RT. Discard supernatant; wash pellet once with 200 μl of 70% ethanol. Centrifuge for 1 min as described above. Remove supernatant completely using a pipette. Dry pellet by incubation of the open reaction tube for 5 min at RT. The samples should not be dried in a speed vacuum! Dissolve pellet in 8 μl Tris-buffer (pH 8.5) and place on ice.

### Example 1F

### Amplification by in Vitro-Transcription

| In vitro transcription: | |
|---|---|
| Template DNA | 8 μl |
| ATP/CTP/GTP/UTP (75 mM each) | 2 μl |
| 10 × buffer | 2 μl |
| T7 RNA polymerase | 2 μl |

Thaw all components and mix them at room temperature, and not on ice, because the spermidine component of the reaction buffer would induce precipitation of the template. Use 0.5 ml or 0.2 ml RNase-free PCR tubes for this step.

Incubate the transcription reaction overnight at 37° C. either in a thermocycler with heated lid (at 37° C.) or in a hybridisation oven. Load 1-2 μl of the reaction mix onto a 1.5% native agarose gel. Add 1 μl DNase to the remaining reaction and incubate for further 15 min at 37° C. To purify the RNA, use the RNeasy kit from Qiagen according to the manufacturer's protocol for RNA-clean-up. At the end of the clean-up procedure, elute the RNA by using 2×50 μl DEPC-water and perform an ethanol precipitation as described above in step 6. Dissolve RNA pellet in 5 μl DEPC water.

The RNA is now ready for labelling and use in a microarray hybridisation or for further amplification by a second amplification round.

### Example 2

### Second Amplification Round (See, e.g., FIG. 1c)

### Example 2A

### Reverse Transcription of Amplified RNA with the Box Primer

| First strand-DNA-synthesis | |
|---|---|
| RNA of the fist amplification round | 4 μl |
| Box primer (5 pmol/μl) | 2 μl |
| dNTP-Mix (10 mM) | 1 μl |
| DEPC-H₂O | 2 μl |

Incubate 4 min at 65° C. in a thermocycler with a heated lid, then place immediately on ice.

| Mastermix for synthesis of the first strand cDNA | |
|---|---|
| 5 × RT-buffer | 4 μl |
| RNase-Inhibitor (20 U/μl) | 1 μl |
| DEPC-H₂O | 5 μl |

Pipette components for the mastermix on ice and add to the tube containing the reverse transcription mix. Place samples in a thermocycler (preheated to 48° C.)

Incubate as follows:
48° C./1 minute
cool to 45° C.
add 1 μl reverse transcriptase (5 U/μl) to each sample
45° C./30 minutes
70° C./15 minutes (enzyme inactivation)

Place samples on ice.

### Example 2B

### RNA Removal

| Removal of RNA from the reaction | |
|---|---|
| First strand cDNA mix | 20 μl |
| RNase-Mix (RNase H/RNase I; each at 5 U/μl) | 1 μl |

Incubate for 20 min at 37° C., hereafter place samples on ice. RNase A was not used for RNA elimination, because RNase A is not readily inactivated. RNase I on the other hand, the enzyme used in this invention, can be inactivated easily and completely by incubation at 70° C. for 15 min.

### Example 2C

### Double-Stranded Template DNA with T7-(dT)₁₈V Primer

| Template DNA from first.strand cDNA with T7-(dT)₁₈V primer | |
|---|---|
| First strand-cDNA | 21 μl |
| dNTP-mix (10 mM) | 1 μl |
| T7-(dT)₁₈V primer (10 pmol/μl) | 2 μl |
| 10 × polymerase buffer | 6 μl |
| H₂O | 20 μl |

Incubation:
96° C./1 minute
42° C./1 minute
add 1 μl reverse transcriptase (5 U/μl) to each sample
42° C./30 minutes

Generation of blunt ends in dsDNA
Cool samples to 37° C.
add 1 μl T4 DNA polymerase (10 U/μl) to each sample
37° C./3 minutes
96° C./15 minutes

Place samples on ice

### Example 2D

### Purification of cDNA with High-Pure PCR Purification Kit (Roche)

| cDNA purification | |
|---|---|
| Reaction mix | 50 μl |
| Binding-buffer | 250 μl |
| Carrier (cot-1-DNA, 100 ng/μl) | 3 μl |

Transfer mix onto provided columns, spin in a tabletop centrifuge at maximal rpm for 1 min. Discard the flow-through. Add 500 μl washing buffer to the column and spin as above, discard flow-through and repeat the wash step with 200 μl washing buffer. Transfer columns onto a new 1.5 ml reaction tube add 50 μl elution buffer, incubate for 1 min at RT and centrifuge as described above. Repeat the elution step once, again using 50 μl buffer.

### Example 2E

### Ethanol Precipitation of Purified cDNA

Do not vortex the Pellet Paint™-carrier stock solution and store in the dark. Keep at −20° C. for long term storage, smaller aliquots can be stored for approximately 1 month at 4° C.

| Ethanol precipitation | |
|---|---|
| Eluate | 100 μl |
| Carrier (Pellet Paint ™) | 2 μl |
| Sodium acetate | 10 μl |
| Ethanol; absolute | 220 μl |

Mix thoroughly (do not vortex) and pellet cDNA by centrifugation at maximal rpm for 10 min at RT.

Discard supernatant; wash pellet once with 200 μl of 70% ethanol. Centrifuge for 1 min as described above. Remove supernatant completely using a pipette. Dry pellet by incubation of the open reaction tube for 5 min at RT. The samples should not be dried in a speed vacuum! Dissolve pellet in 8 μl Tris-buffer (pH 8.5) and place on ice.

### Example 2F

### Second Amplification by In Vitro-Transcription

| In vitro transcription: | |
|---|---|
| Template DNA | 8 μl |
| ATP/CTP/GTP/UTP (75 mM each) | 2 μl |
| 10x buffer | 2 μl |
| T7 RNA polymerase | 2 μl |

Thaw all components and mix them at RT, and not on ice, because the spermidine component of the reaction buffer would induce precipitation of the template. Use 0.5 ml or 0.2 ml RNase-free PCR tubes for this step.

Incubate the transcription reaction overnight at 37° C. either in a thermocycler with heated lid (at 37° C.) or in a hybridisation oven. Load 1-2 μl of the reaction mix onto a 1.5% native agarose gel. Add 1 μl DNase to the remaining reaction and incubate for further 15 min at 37° C. To purify the RNA, use the RNeasy kit from Qiagen according to the manufacturer's protocol for RNA-clean-up. At the end of the clean-up procedure, elute the RNA by using 2×50 μl DEPC-water and perform an ethanol precipitation as described above in step 6. Dissolve RNA pellet in 5 μl DEPC water.

The RNA is now ready for labelling and use in a microarray hybridisation or for further amplification by a third amplification round (a third amplification round is exactly performed as described in Examples 2A-2F).

1-37. (canceled)

38. A method for the amplification of ribonucleic acids comprising the following steps:
a) a single stranded DNA is produced from an RNA by means of reverse transcription, using a single-stranded primer having a defined sequence, an RNA-dependent DNA polymerase and deoxyribonucleoside triphosphates;
b) the template RNA is removed;
c) a DNA duplex is produced by means of a single-stranded primer, a DNA polymerase and deoxyribonucleoside triphosphates, wherein the a single-stranded primer comprises a box sequence and a sequence of 3 to 9 oligonucleotides of random sequence, wherein the Box sequence is present in the 5′ region of the single stranded primer and comprises a sequence with a length of 10 to 25 nucleotides having a low homology to sequences expressed by organisms from which the RNA to be amplified was obtained;
d) the duplex is separated into single-stranded DNAs;
e) DNA duplexes are produced from one of the single-stranded DNAs obtained in step (d) by means of a single-stranded primer comprising a promoter sequence at its 5′end and the same defined sequence as the primer used in step (a) at its 3′end, a DNA polymerase and deoxyribonucleoside triphosphates;
f) a plurality of single stranded RNAs is produced, both ends of which comprise defined sequences, by means of an RNA polymerase and ribonucleoside triphosphates.

39. The method according to claim 38, wherein the single-stranded RNA obtained have the inverse sense orientation (antisense sequence) in relation to the RNA starting material.

40. The method according to claim 38, characterised in that the single-stranded primer used in step (a) contains an oligo-dT-sequence.

41. The method according to claim 38, characterised in that a 5′-(dT)₁₈V-primer is used in step (a) for reverse transcription, with V being any deoxyribonucleotide-monomer apart from dT.

42. The method according to claim 38, characterised in that in step (b) the RNA is hydrolysed by means of RNase.

43. The method according to claim 38, characterised in that in step (b) the RNA is removed by means of RNase I and/or RNase H.

44. The method according to claim 38, characterised in that in step (c) a single-stranded primer is used with the following sequence: GCA TCA TAC AAG CTT GGT ACC NNN NNN TCT (30 nt).

45. The method according to claim 38, characterised in that a reverse transcriptase is used as DNA polymerase.

46. The method according to claim 38, characterised in that dATP, dCTP, dGTP and dTTP are used as deoxyribonucleotide-monomers.

47. The method according to claim 38, characterised in that in step (d) DNA double strands are separated in single strands by means of heat.

48. The method according to claim 38, characterised in that in step (e) a single-stranded primer is used, which comprises the sequence of either the T7, T3 or SP6 RNA polymerase.

49. The method according to claim 38, characterised in that in step (e) a single-stranded primer is used, containing not only a promoter sequence but also an oligo(dT)-sequence of at least 8 nucleotides.

50. The method according to claim 38, characterised in that in step (e) the single-stranded primer has the following sequence: ACT AAT ACg ACT CAC TAT A g⁺¹ g (dT)₁₈V (40 nt).

51. The method according to claim 38, characterised in that in step (f) T7 RNA polymerase is used as RNA polymerase

52. The method according to claim 38, characterised in that ATP, CTP, GTP and UTP are used as ribonucleotide-monomers.

53. The method according to claim 38, characterised in that the amplification factor of the starting RNA sequence is at least 500, preferably more than 1000.

54. The method according to claim 38, characterised in that the method comprises after step (f) the following steps for further amplification of ribonucleic acids:
g) using the in step (f) generated single-stranded RNAs as template, single-stranded DNA is synthesised using reverse transcriptase, a single-stranded primer, containing the Box sequence, an RNA-dependant DNA polymerase and deoxyribonucleoside triphosphates;
h) the RNA is removed;
i) using the in (h) generated single-stranded DNA as template, double-stranded DNA is synthesised using a single-stranded primer, comprising a promoter sequence in its 5′ region and the same defined sequence as the primer used in step (a), in its 3′ region, a DNA polymerase and deoxyribonucleoside triphosphates;
j) a multitude of single-stranded RNAs is synthesized using a RNA polymerase and ribonucleoside triphosphates.

55. The method according to claim 54, characterised in that in step (i) the single stranded primer is identical with the single-stranded primer used in step (e).

56. The method according to claim 54, characterised in that in step (h) the RNA is hydrolysed by means of RNase.

57. The method according to claim 54, characterised in that all single-stranded RNAs produced in step (O) have inverse orientation.

58. A kit for ribonucleic acid amplification according to the method of claim 38, comprising the following components:
a) at least at least one single-stranded primer comprising a promoter sequence;
b) at least one single-stranded primer comprising a box sequence;
c) an RNA-dependent DNA polymerase;
d) deoxyribonucleoside triphosphates;
e) a DNA-dependent DNA polymerase;
f) an RNA polymerase; and
g) ribonucleoside triphosphates.

59. The kit according to claim 58, characterised in that the kit comprises three different single-stranded primers.

60. The kit according to claim 58, characterised in that the single-stranded primer comprising the promoter sequence, also comprises an oligo-dT-sequence.

61. The kit according to claim 58, characterised in that a single-stranded primer comprises a 5′-(dT)₁₈V-primer sequence for reverse transcription, with V being any deoxyribonucleotide-monomer apart from dT.

62. The kit according to claim 58, characterised in that in addition, the kit comprises RNase I and/or RNase H.

63. The kit according to claim 58, characterised in that the kit comprises a single-stranded primer with a T7, T3 or SP6 RNA polymerase promoter sequence.

64. The kit according to claim 58, characterised in that a single-stranded primer is used with the following sequence: ACT AAT ACg ACT CAC TAT A g⁺¹ g (dT)₁₈V (40 nt).

65. The kit according to claim 58, characterised in that it comprises a reverse transcriptase as DNA polymerase.

66. The kit according to claim 58, characterised in that it comprises the T7 RNA polymerase.

67. The kit according to claim 58, characterised in that it comprises a composition for labelling of DNA with a detectable moiety.

68. The kit according to claim 58, characterised in that the kit includes a DNA-microarray.

69. A method for nucleic acid analysis that involves production of ribonucleic acids, amplification with the method according to claim 38, and analysis by means of microarrays.

70. The method according to claim 69, characterised in that the ribonucleic acids is isolated from a biological sample.

71. The method according to claim 69, characterised in that ribonucleic acids are amplified, converted to cDNA by means of reverse transcription, and the cDNAs are analysed by means of micoarrays.

72. The method according to claim 69, characterised in that the amount and/or sequence of the cDNA are analysed.
